(19) United States
(12) Patent Application Publication
(10) Pub. No.: US 2020/0058396 A1
(43) Pub. Date: Feb. 20, 2020

(54) REMOTELY MANAGING HEALTHCARE UTILIZING PERSONAL ASSISTANT DEVICES

(71) Applicant: CERNER INNOVATION, INC., Kansas City, KS (US)

(72) Inventors: Chad G. Hays, Overland Park, KS (US); Damon M. Herbst, Shawnee, KS (US); Randolph S. Lantz, Parkville, MO (US)

(21) Appl. No.: 16/545,544

(22) Filed: Aug. 20, 2019

Related U.S. Application Data
(60) Provisional application No. 62/719,965, filed on Aug. 20, 2018.

(51) Int. Cl. G16H 40/67; G16H 80/00

(52) U.S. Cl. CPC G16H 40/67; G16H 80/00

(57) ABSTRACT

Systems and methods for utilizing personal assistant devices to remotely manage healthcare are presented. In some aspects a personal assistant device associated with a patient (e.g., an AMAZON ECHO or a GOOGLE HOME device) is utilized to audibly output at least one instruction to be performed by the patient. In aspects, the instruction is to be performed by the user utilizing a connected health marker monitoring device. Upon the patient performing the instruction, for instance, upon a measurement being received from the connected health marker monitoring device, the result of the instruction (e.g., the measurement) is transmitted to one or more of an electronic health information system for association with the patient's healthcare records, a device associated with a member of a care team associated with the patient, a data store, and a control component for further evaluation.

### CROSS-REFERENCE TO RELATED APPLICATIONS

This application claims priority to U.S. Provisional Patent Application No. 62/719,965, filed Aug. 20, 2018 and entitled “Remotely Managing Healthcare Utilizing Personal Assistant Devices,” which application is hereby incorporated by reference as if set forth in its entirety herein.

### BACKGROUND

There are numerous chronic healthcare conditions that are best managed with consistent monitoring of various health markers over extended periods of time. For instance, a patient with congestive heart failure may benefit from daily monitoring of health markers such as body weight, blood pressure, and oxygen saturation such that any abnormality or concerning change may be identified as quickly as possible.

In many cases, rather than undergoing the inconvenience of continual visits to a hospital or clinical care office to monitor health markers, patients may be permitted to monitor their health markers from the comfort of their own homes. In some such cases, devices for monitoring various health markers may be provided to a patient for their at-home use. Some such devices are capable of connection, for instance, via BLUETOOTH or the like, with a communications component configured to receive measurements of various health markers and provide such measurements to appropriate parties, for instance, to an Electronic Health Information System (EHIS) having healthcare records associated with the patient. When utilizing such connected in-home devices, upon recording, measurements associated with various health markers automatically may be transmitted to the EHIS for association with the patient's healthcare records.

While generally more convenient than having to attend appointments for monitoring health markers on a consistent basis over the long term, patients may struggle to remember what measurements they are to record on any given day. For instance, a patient may have an order in her chart indicating that she is to monitor her blood pressure and oxygen saturation daily and her weight twice a week. In this instance, on any particular day, she may not be able accurately to remember whether or not she is to record her weight.

Additionally, even when a patient remembers the tasks to be performed, if a measurement is abnormal for any reason such that additional action may be required, the patient may not realize the abnormality. Even if the patient notices the abnormality, she may not know or remember if it rises to the level that a member of her healthcare team should be notified and/or if additional actions may be required. Further, she may not accurately understand what is done with the recorded measurements. For instance, even if a patient realizes the health marker monitoring device is connected with her healthcare records, there still may be a lack of understanding with regard to whether all measurements are provided to her healthcare team, if only those measurements that are abnormal are provided, if the healthcare team conducts a daily review of measurements received, or the like. Thus, she may be left wondering what her next action should be.

### SUMMARY

The present technology generally relates to devices, systems, and methods for using personal assistant devices to remotely manage healthcare. In some cases, at-home health marker monitoring devices may be utilized. In some cases, measurements recorded by the at-home health marker monitoring devices may be integrated with a patient's healthcare records.

In brief, and at a high level, the present disclosure describes technology that may utilize a personal assistant device associated with a patient (e.g., an AMAZON ECHO or a GOOGLE HOME device) to output at least one instruction to be performed by the patient using a connected health marker monitoring device. In some configurations, the at least one instruction may be audibly output by the personal assistant device. Upon the patient performing the instruction, a measurement may be received from the connected health marker monitoring device and transmitted to an Electronic Health Information System (EHIS) for association with the patient's healthcare records. In some configurations, the instruction may be output upon receipt of a voice command from the patient, for instance, a voice command inquiring which tasks the patient is to perform the present day. In some configurations, the instruction may be output upon receipt of a voice command from the patient to receive the content of an alert, the patient being aware of the alert due to an alert indicator associated with the personal assistant device being activated (e.g., illuminated). In the latter configurations, an alert may automatically be transmitted to the personal assistant device associated with the patient on a particular time schedule, may automatically be transmitted due to receipt of an abnormal measurement from a connected health marker monitoring device, may be customized and remotely provided by a member of the patient's healthcare team, or the like. In any event, once the alert is received by the personal assistant device, the alert indicator may be activated.

Accordingly, many of the problems associated with remotely monitoring health markers on a consistent, long-term basis may be solved utilizing this technology. For example, in some instances, either upon request by a patient or in response to prompting via an alert indicator, a personal assistant device may aid the patient in remembering the tasks associated with his or her healthcare management that are to be performed on a given day. In some instances, the personal assistant device, upon receiving a measurement from a connected health marker monitoring device, may output a message for the patient informing the patient of the value of the measurement recorded by the connected health marker monitoring device. By way of example, if the patient engages with a connected weight scale, upon recording the patient's weight, the personal assistant may output a message stating: “Your weight is 185 pounds.” In some instances, the personal assistant device, upon receiving a measurement from a connected health marker monitoring device, may output a message informing the patient of the action to be taken with that measurement. For instance, if the patient engages with a connected weight scale, upon recording the patient's weight, the personal assistant may output a message stating: “Your weight has been recorded and sent to a member of your healthcare team for review.”

Further, in some instances, upon determining that a recorded measurement is abnormal (e.g., automatically determining based upon risk algorithms or determined by a healthcare team member upon receipt of the recorded measurement), the personal assistant device may output further instructions to be performed by the patient. In some instances, the patient may be informed of the abnormal measurement and, thus, of the reason why additional action is requested. For instance, if the patient engages with a connected weight scale, upon recording the patient's weight, the personal assistant may output a message stating: “Your weight is 185 pounds. I noticed that your weight has increased abnormally over the last forty-eight hours. Based on your diagnosed condition, I have alerted your healthcare team about this. May I ask you some follow-up questions?”

One aspect of the present technology that may accomplish these objectives is a system for utilizing personal assistant devices to remotely manage healthcare. The system may include a personal assistant device associated with a patient and having a listening component and a speaker, the personal assistant device being configured to receive voice commands from the patient and to provide audible instructions to the patient. The system further may include a processor in communication with the personal assistant device and an EHIS. Still further, the system may include one or more computer-readable media storing computer-readable instructions that, when executed by the processor, cause the processor to: receive, via the listening component, a voice command from the patient, transmit the voice command to the EHIS, receive, from the EHIS, a response instruction to the voice command, and audibly output, via the speaker, the response instruction.

In another aspect of the present technology, a system for utilizing personal assistant devices to remotely manage healthcare is provided. The system may include a personal assistant device associated with a patient and having a listening component, a speaker, and an alert indicator, the personal assistant device being configured to receive voice commands from the patient and to provide audible instructions to the patient. The system further may include a connected health marker monitoring device associated with the patient. Still further, the system may include a processor in communication with the personal assistant device, the connected health marker monitoring device and an EHIS. Additionally, the system may include one or more computer-readable media storing computer-readable instructions that, when executed by the processor, cause the processor to: receive, from the EHIS, an alert associated with the patient, the alert having content that includes at least one task to be performed by the patient using the connected health marker monitoring device; activate the alert indicator associated with the personal assistant device; receive, via the listening component, a voice command from the patient requesting the content of the alert; and audibly output the alert content via the speaker.

In yet another aspect of the present technology, a method for utilizing personal assistant devices to remotely manage healthcare is provided. The method includes audibly outputting, via a speaker of a personal assistant device associated with a patient, at least one instruction to be performed by the patient using a health marker monitoring device; receiving, at a control component, a measurement from the health marker monitoring device, the measurement being taken in response to the patient performing the at least one instruction; and transmitting the measurement, via the control component, to an EHIS.

Additional objects, advantages, and novel features of the technology are described below in the Detailed Description. They will, in part, become apparent to those skilled in the art, or they may be learned by practice of the technology.

### BRIEF DESCRIPTION OF THE DRAWINGS

The present technology is described in the Detailed Description with reference to the attached figures, wherein:

FIG. 1 is a block diagram of an exemplary computing environment suitable for us in implementing aspects of the technology described herein;

FIG. 2 is a block diagram of an exemplary system for remotely managing healthcare in accordance with an aspect hereof;

FIGS. 3-5 are block diagrams of exemplary methods for remotely managing healthcare in accordance with aspects hereof; and

FIGS. 6A-6V present a computer-readable code for handling exemplary intents described herein in accordance with aspects hereof.

### DETAILED DESCRIPTION

The subject matter of the present technology is described herein with specificity to meet statutory requirements. However, the description itself is not intended to limit the scope of this disclosure. Rather, the inventors have contemplated that the claimed subject matter also might be embodied in other ways, to include different steps or combinations of steps similar to the ones described in this document, in conjunction with other present or future technologies. Moreover, although the term “step” may be used herein to connote different elements of methods employed, the term should not be interpreted as implying any particular order among or between various steps disclosed, unless the order of individual steps is explicitly described.

Embodiments of the present technology are directed to methods, systems, and computer-readable media for utilizing personal assistant devices to remotely manage healthcare. Certain aspects may provide for audibly outputting, from a speaker associated with a personal assistant device, at least one instruction to be performed by a patient using a connected health marker monitoring device (e.g., a health marker monitoring device that is communicatively coupled with an Electronic Health Information System (EHIS) associated with healthcare records of the patient). Upon the patient performing the instruction, a measurement may be received from the connected health marker monitoring device and transmitted, via a processor, to the EHIS for association with the patient's healthcare records. In embodiments, the at least one instruction may be audibly output upon receiving, via a listening component associated with the personal assistant device (e.g., a microphone), a voice command from the patient, for instance, a voice command inquiring which tasks the patient is to perform the present day. In embodiments, the instruction may be audibly output upon receiving a voice command from the patient to receive the contents of an alert, the patient being aware of the alert via activation (e.g., illumination) of an alert indicator associated with the personal assistant device. Further aspects and embodiments are described in detail below.

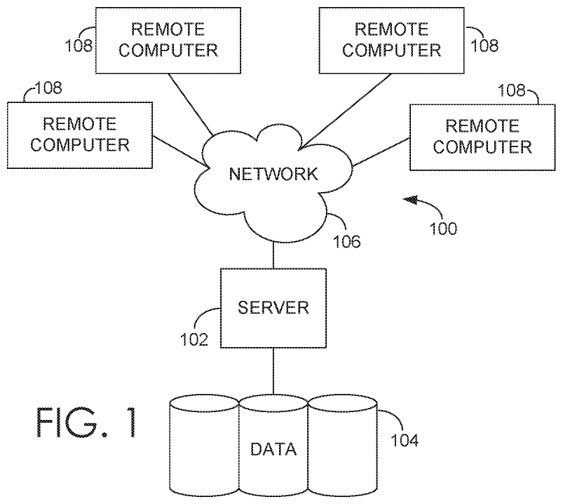

With reference now to FIG. 1, an exemplary computing environment suitable for use in implementing aspects of the technology is described below. An exemplary computing environment (e.g., a healthcare-information computing-system environment) with which embodiments may be implemented is provided. The computing environment is merely an example of one suitable computing environment and is not intended to suggest any limitation as to the scope of use or functionality of the technology. Neither should the computing environment be interpreted as having any dependency or requirement relating to any single component or combination of components illustrated therein.

The present technology may be operational with numerous other purpose computing system environments or configurations. Examples of well-known computing systems, environments, and/or configurations that may be suitable for use with aspects of the present invention include personal computers, server computers, hand-held or laptop devices, multiprocessor systems, microprocessor-based systems, set top boxes, programmable consumer electronics, network PCs, minicomputers, mainframe computers, personal assistant devices, distributed computing environments that include any of the above-mentioned systems or devices, and the like.

The present technology may be described in the general context of computer-executable instructions, such as program modules, being executed by a computer. Exemplary program modules include routines, programs, objects, components, and data structures that perform particular tasks or implement particular abstract data types. The present technology may be practiced in distributed computing environments where tasks are performed by remote processing devices that are linked through a communications network. In a distributed computing environment, program modules may be located in association with local and/or remote computer storage media (e.g., memory storage devices).

The computing environment comprises a computing device in the form of a control server 102. Exemplary components of the control server comprise a processing unit, internal system memory, and a suitable system bus for coupling various system components, including data stores, with the control server. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The control server 102 typically includes, or has access to, a variety of non-transitory computer-readable media. Computer-readable media can be any available media that may be accessed by the control server 102, and includes volatile and nonvolatile media, as well as, removable and non-removable media. By way of example, and not limitation, computer-readable media may comprise computer storage media and communication media. Computer storage media includes volatile, nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above also should be included within the scope of computer-readable media.

The control server 102 may operate in a computer network using logical connections to one or more remote computers 108. The remote computers 108 may be located at a variety of locations including operating systems, device drivers and healthcare information workflows. The remote computers also may be physically located in traditional and nontraditional healthcare care environments so that the entire healthcare community may be capable of integration on the network. The remote computers might be personal computers, servers, routers, network PCs, peer devices, other common network nodes, or the like and might comprise some or all of the elements described above in relation to the control server 102. The devices can be personal digital assistants or other like devices.

The computer network 106 may comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof may be stored in association with the control server 102, the data store 104, or any of the remote computers 108. For example, various application programs may reside on the memory associated with any one or more of the remote computers 108. It will be understood and appreciated by those of ordinary skill in the art that the network connections shown are exemplary and other means of establishing a communications link between the computers (e.g., the control server 102 and the remote computers 108) may be utilized.

In operation, an organization may enter commands and information into the control server 102 or convey the commands and information to the control server 102 via one or more of the remote computers 108 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information also may be sent directly from a remote health marker monitoring device to the control server 102. In addition to a monitor, the control server 102 and/or the remote computers 108 may comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

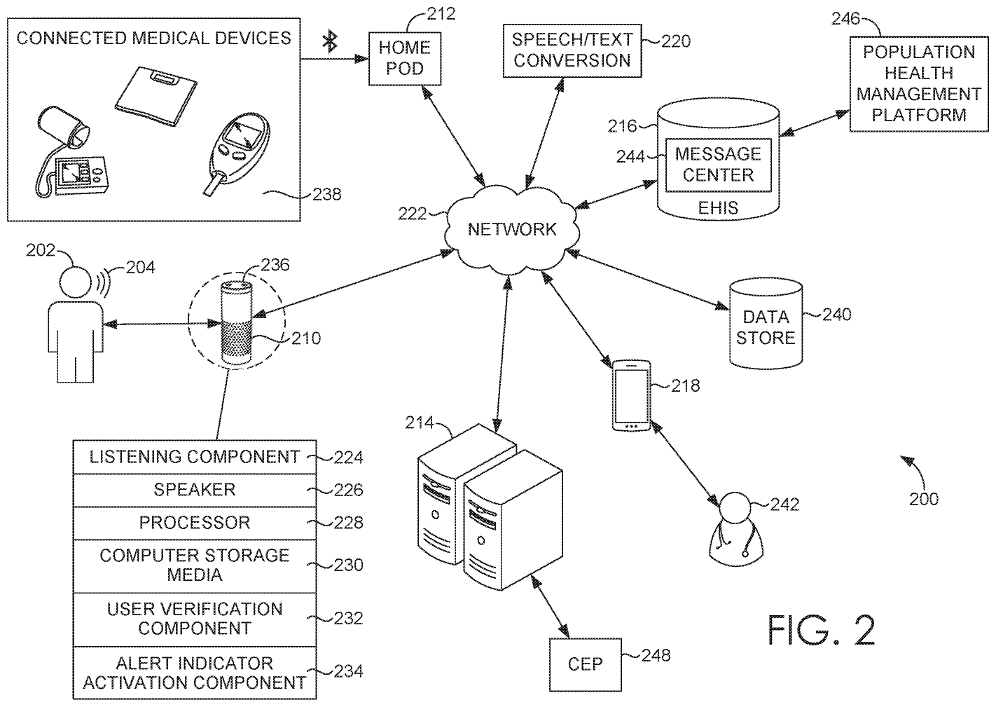

Turning now to FIG. 2, illustrated is a block diagram of an exemplary system 200 for remotely managing healthcare. In the illustrated aspect, the system comprises a personal assistant device 210, a connected device communications component 212, a control component 214, an Electronic Health Information System (EHIS) 216, a care team member device 218, and a speech/text conversion component 220, all in communication with one another through a network 222. The network 222 may be any type of communication scheme that allows devices to exchange data. For example, the network 222 may include fiber optic, wired and/or wireless communication capability in any of a plurality of protocols, such as TCP/IP, Ethernet, WAP, IEEE 802.11, or any other protocols. Implementations are contemplated in which the system 200 may be accessible through a shared public infrastructure (e.g., the Internet), an extranet, an intranet, a virtual private network (VPN), a local area network (LAN), a wide area network (WAN), a peer-to-peer (P2P) network, a wireless communications network, a telephone network, a facsimile network, a cloud network, or any combination thereof. Such networks are commonplace in today's computing environments and, accordingly, are not further described herein. Although many of the components illustrated in FIG. 2 are described as individual components or functional entities, in some cases, they may be implemented as discrete or distributed components, or in conjunction with other components, having any suitable location or combination.

The system 200 includes a personal assistant device 210. Personal assistant devices are a category of smart speakers that enable users to speak voice commands to interact with a personal assistant service that aids the user in obtaining answers to questions and/or in completing certain tasks. Exemplary personal assistant devices include, without limitation, AMAZON ECHO which utilizes a personal assistant service called “Alexa” and GOOGLE HOME, which utilizes a personal assistant service called “Google Assistant.” As illustrated, the personal assistant device 210 includes a listening component 224, a speaker 226, a processor 228, computer storage media 230, a user verification component 232 and an alert indicator activation component 234. The listening component 224 may be configured for detecting an audible signal 204, for example a voice command from a user 202. One common example of a type of listening component 224 is a microphone. In some aspects, the listening component 224 may be a separate component that is communicatively coupled, for example, by hardwire or wireless, to the personal assistant device 210.

The audible signal 204 may be detected from a user 202. In general, any person could be considered a user of the remote healthcare management system 200. In a non-limiting example, the user 202 may be a patient, for instance, a patient having at least one chronic health condition for which there is a desire to engage in the monitoring of one or more health markers over time. The audible signal 204 may be a voice command, such as a command for the user 202 to be notified of tasks he or she is to perform or a command for the user 202 to be notified of a reason for activation of an alert indicator 236 associated with the personal assistant device 210 (more fully described below). In some aspects, the audible signal 204 may be in the form of a question.

In some aspects, the initial sound of the audible signal 204 may include a “wake word,” that is, a word that alerts the personal assistant device 210 that the user 202 is speaking a voice command intended for the device 210. By way of non-limiting example, the wake word generally utilized with the AMAZON ECHO personal assistant device is “Alexa” and the wake word generally utilized with the GOOGLE HOME personal assistant device is “Google.” In a non-limiting example, after the wake word signals the personal assistant device 210 that a request intended for the device is to follow, the user may speak an utterance (e.g., a statement) to invoke their intent. As used in this context, an “intent” represents an action that fulfills the user's spoken request.

By way of example, assume the user 202 is utilizing an AMAZON ECHO personal assistant device and that she desires to have the personal assistant device inform her of her tasks to perform for the day. The user 202 in this instance may speak one of a number of utterances that the personal assistant device is programmed to recognize as associated with the “GetTasks” intent. For instance, the user may in this instance utter “What are my tasks for the day?” or “What are my tasks for today?”. In some instances, intents can have “slots” that the user may customize to request fulfillment of a particular request. For instance, utilizing the above example, the user 202 may utter “What are my task for {Date}?” In this instance, the user would speak the current date or a future in the “slot” indicated as {Date}. If the user 202 desired to be reminded of tasks for a past date, the user 202 similarly may utter “What were my tasks for {Date}?”. An exemplary response from the personal assistant device 210 in each of these instances may be “You have the following tasks to perform: 1) Weigh yourself. 2) Take your blood pressure.”

The user 202 may utilize a similar spoken format to fulfill other intents as well. For instance, if the user desired to find out what the measurement associated with a certain date and measurement recorded by a connected health monitoring device, she could utter any of the following utterances that the personal assistant device would be programmed to recognize to fulfill the “GetMeasurement” intent: “What was my {Measurement}{Date}?”, “What my {Measurement} was {Date}?” or “Tell me my {Measurement} from {Date},” and the like. In each instance, the words spoken in place of the {Measurement} and {Date} slots customize the utterance to aid the user in attaining the desired request fulfillment. An exemplary response from the personal assistant device 210 in this instance would depend upon the connected health marker monitoring device and date. By way of example only, an exemplary response may be “Your weight yesterday was 185 pounds.”

Similarly, the user may utilize the referenced format to find out if a particular measurement recorded from a connected health marker monitoring device was abnormal or outside of a reference range. Such a measurement could be, by way of example only, body weight, blood pressure, blood sugar level, and the like. In this instance, the user may utter any of the following utterances that the personal assistant device would be programmed to recognize to fulfill the “GetAbnormality” intent: “Was my {Measurement} abnormal {Date}?”, “Tell me if my {Measurement} was abnormal {Date},” {Measurement} {Date} was normal,” “{Measurement} {Date} was high”, {Measurement} {Date} was low,” and the like. In each instance, the words spoken in place of the {Measurement} and {Date} slots customize the utterance to aid the user in attaining the desired request fulfillment. An exemplary response from the personal assistant device 210 in this instance would depend upon the connected health marker monitoring device and date. By way of example only, an exemplary response may be: “Your weight today of 195 pounds is high. Normal weight values for your age and demographic range between 155 pounds and 185 pounds.”

In yet another example, the user could utilize the referenced format to inquire of her current diagnosis. In this instance, the user 202 may speak any of the following utterances that the personal assistant device would be programed to recognize to fulfill the “GetDiagnosis” intent: “Tell me my diagnosis,” “What have I been diagnosed with?”, “What my diagnosis is,” and the like. An exemplary response from the personal assistant device 210 may include the name of a chronic condition with which the user 202 has been diagnosed and, if desired, a brief explanation thereof.

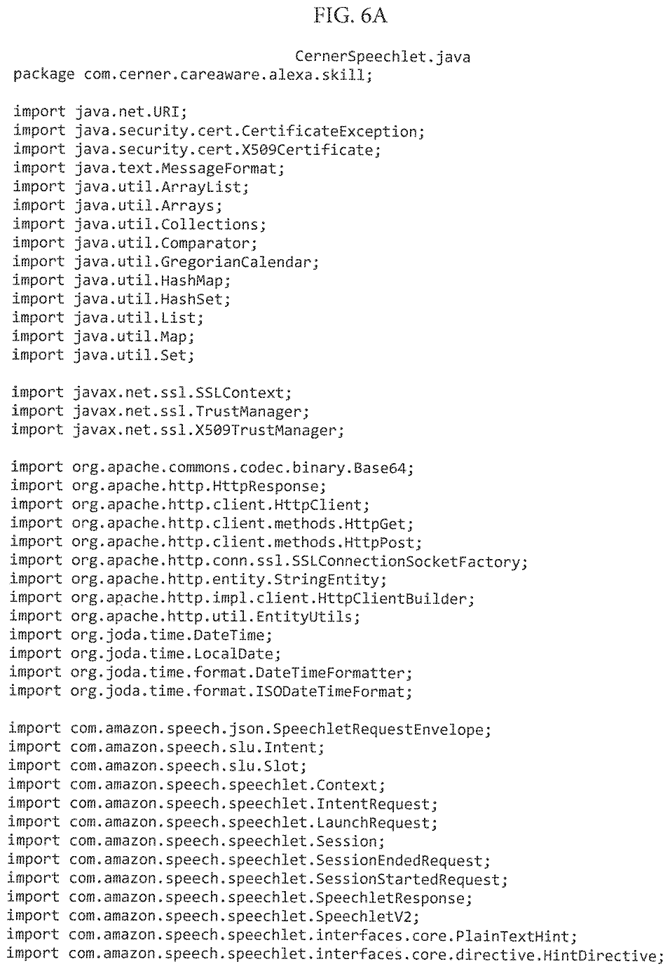

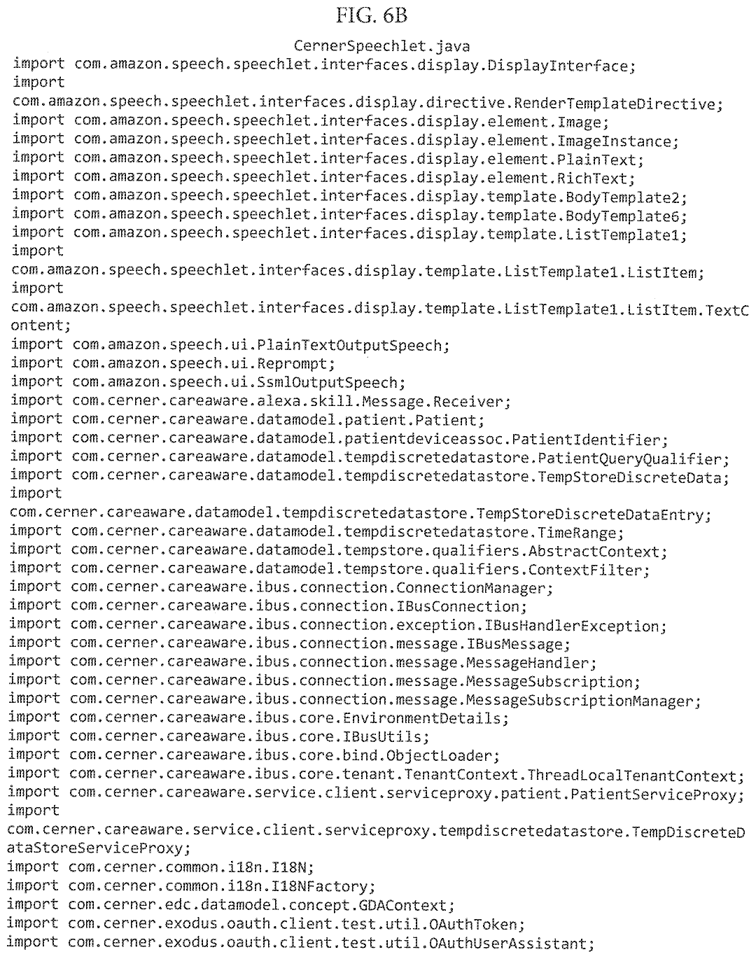

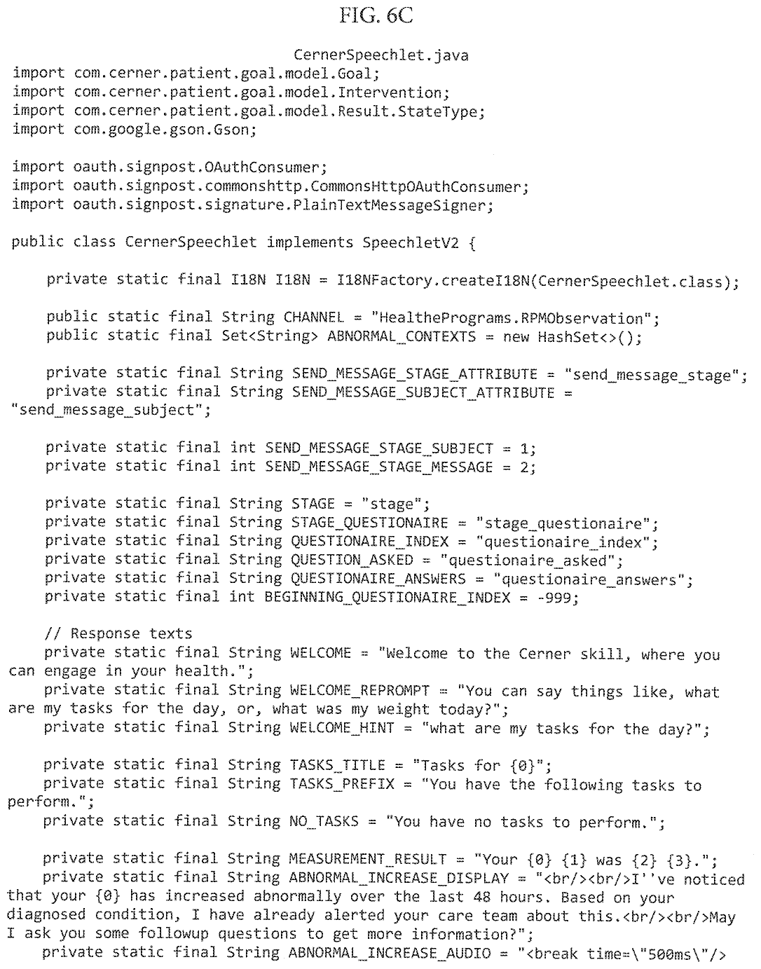

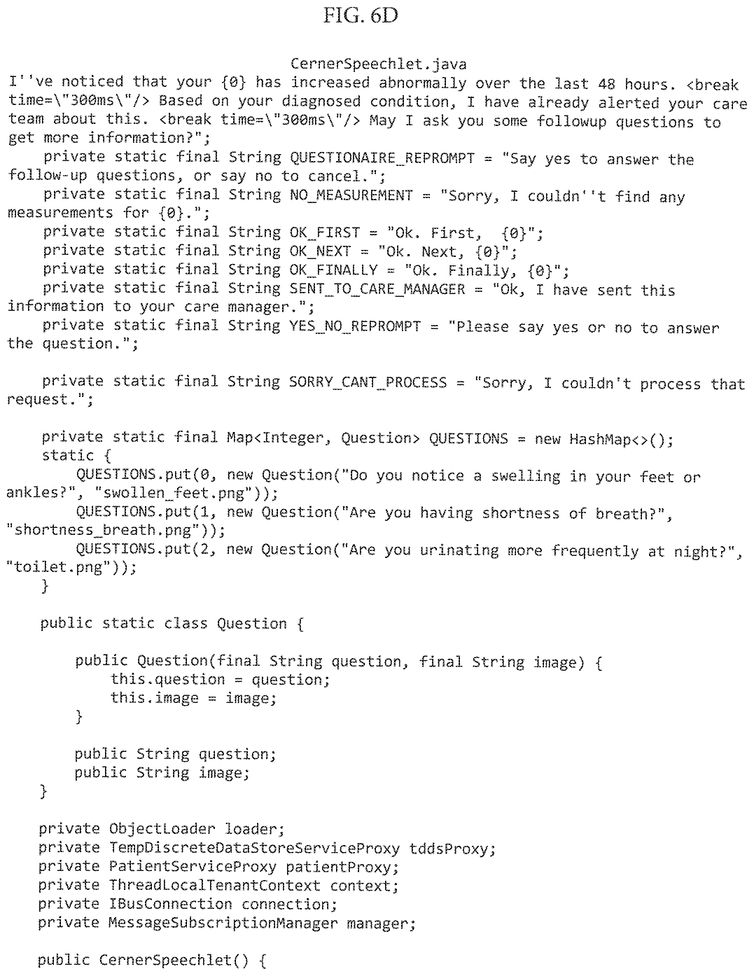

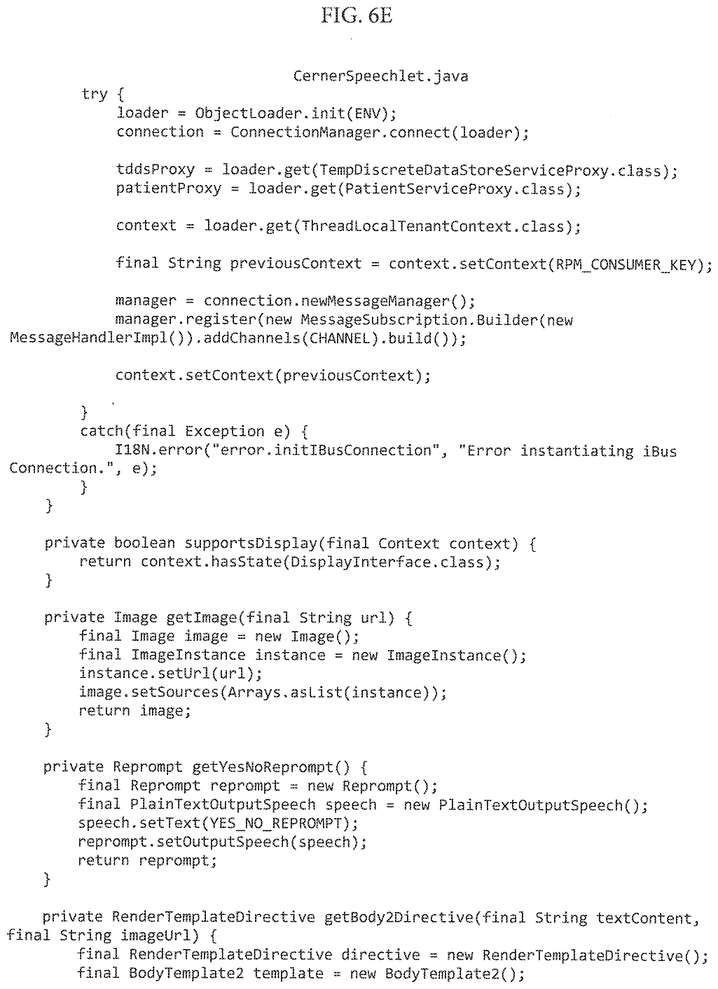

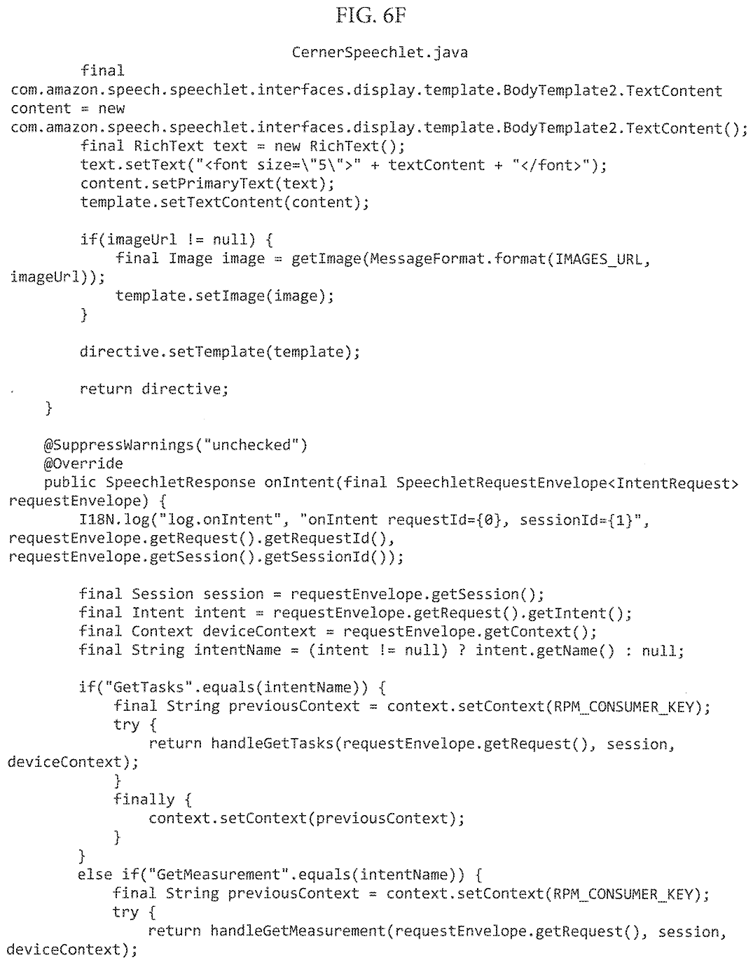

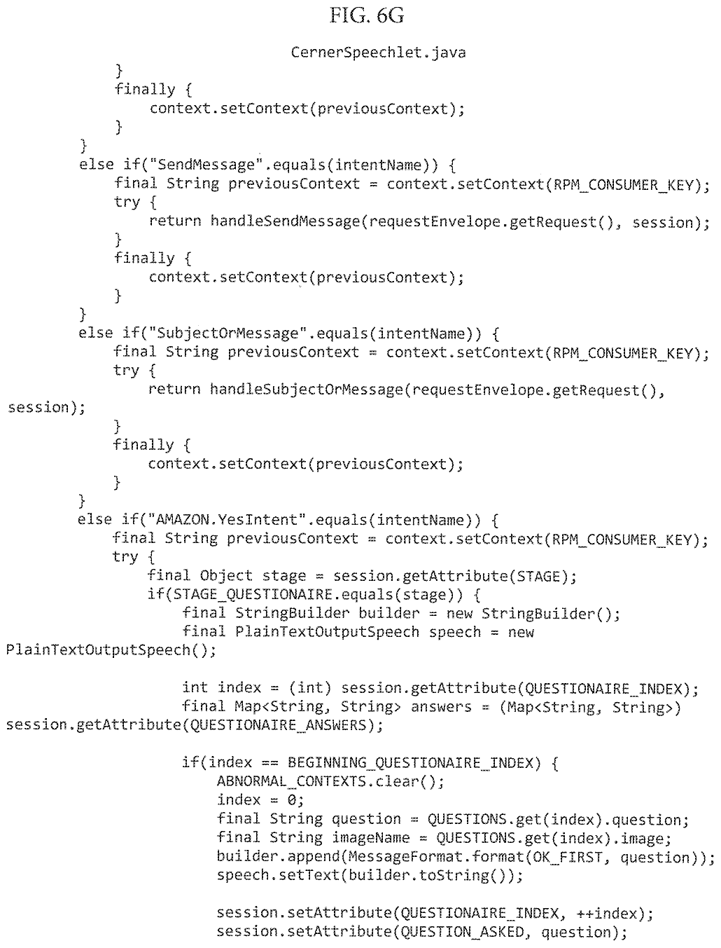

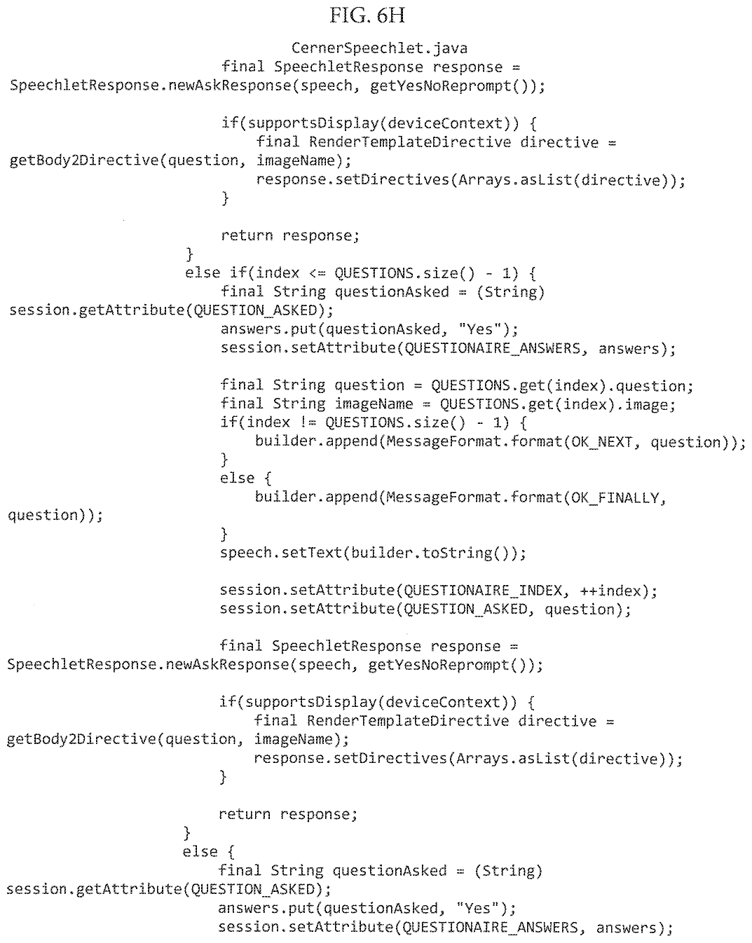

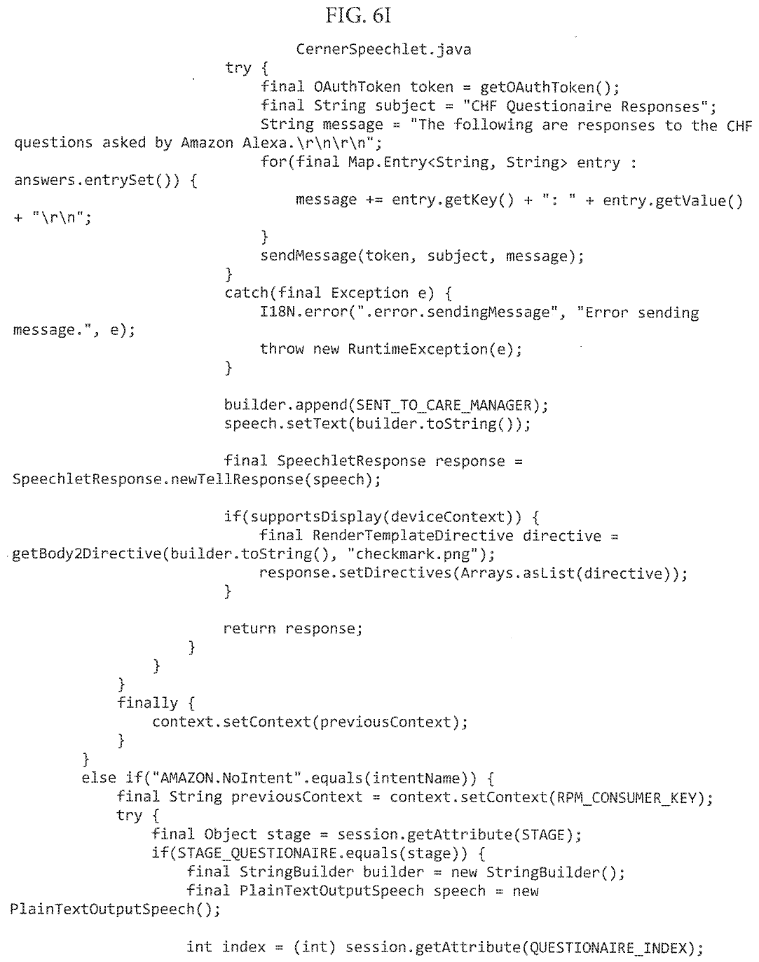

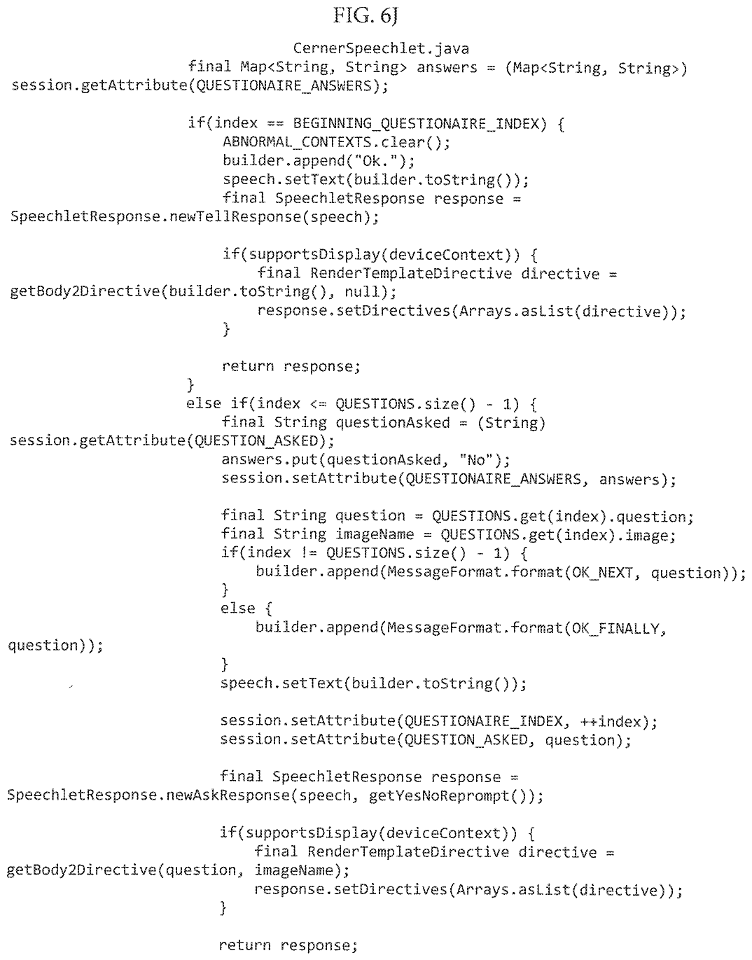

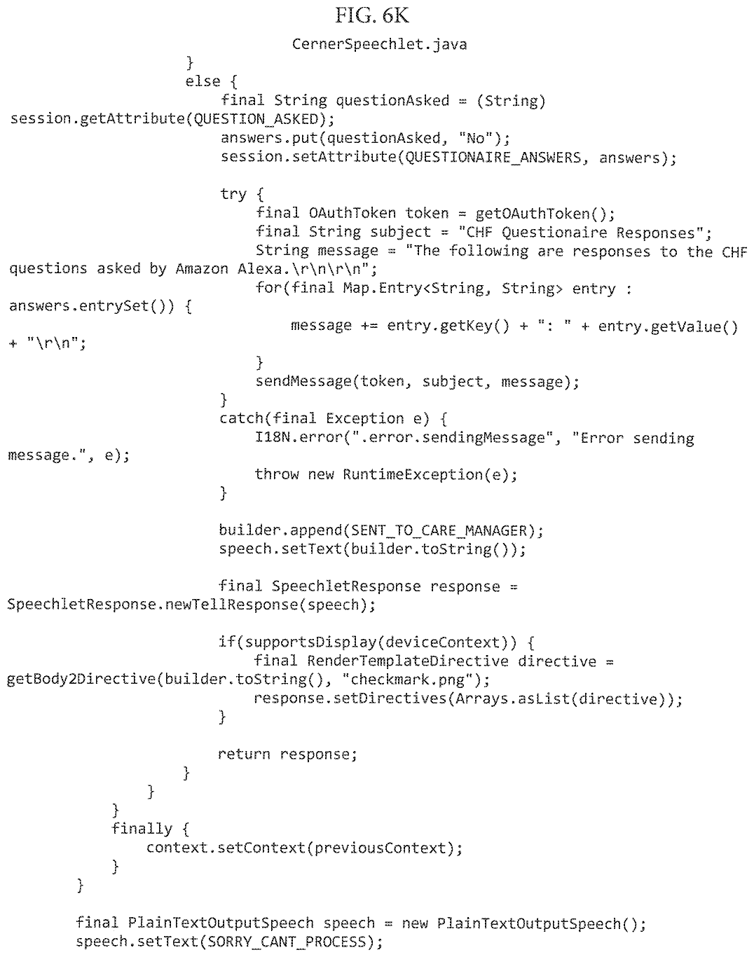

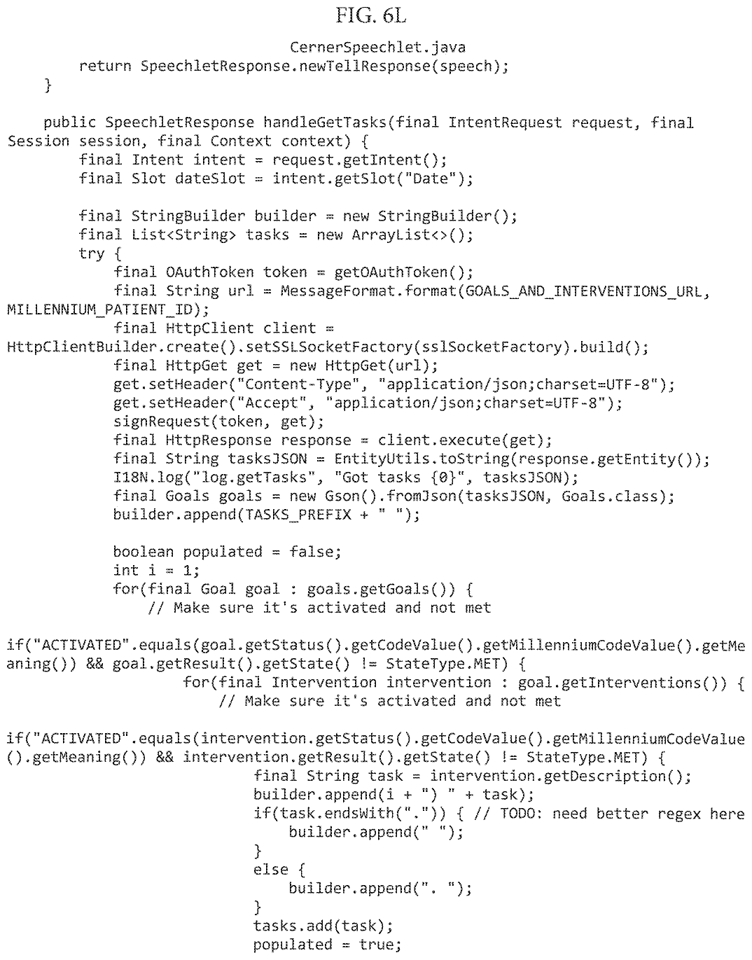

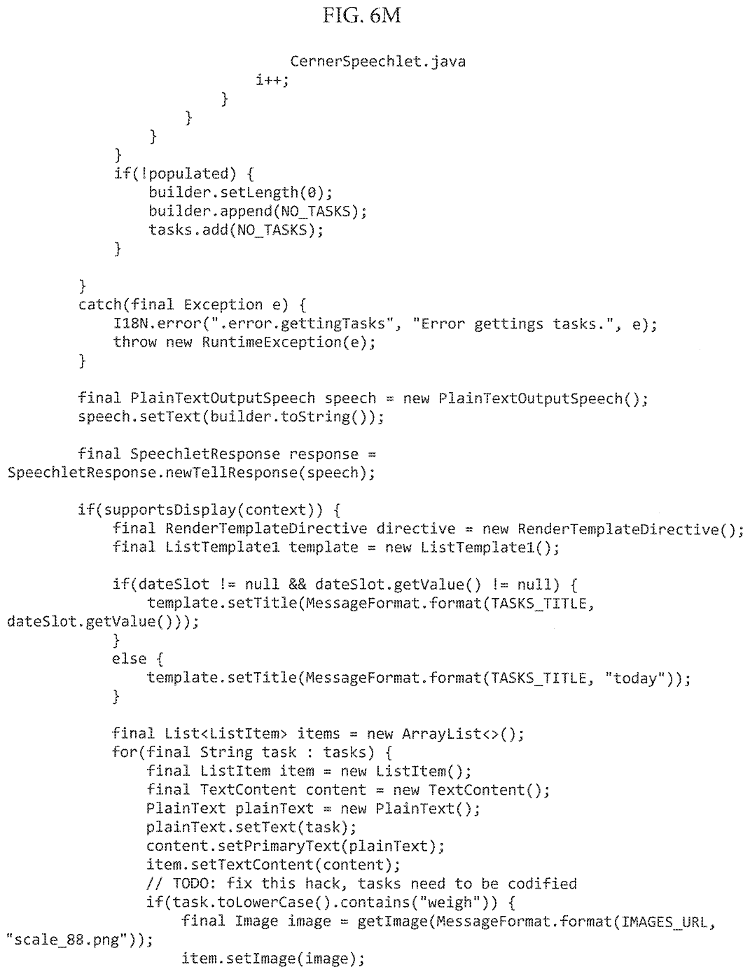

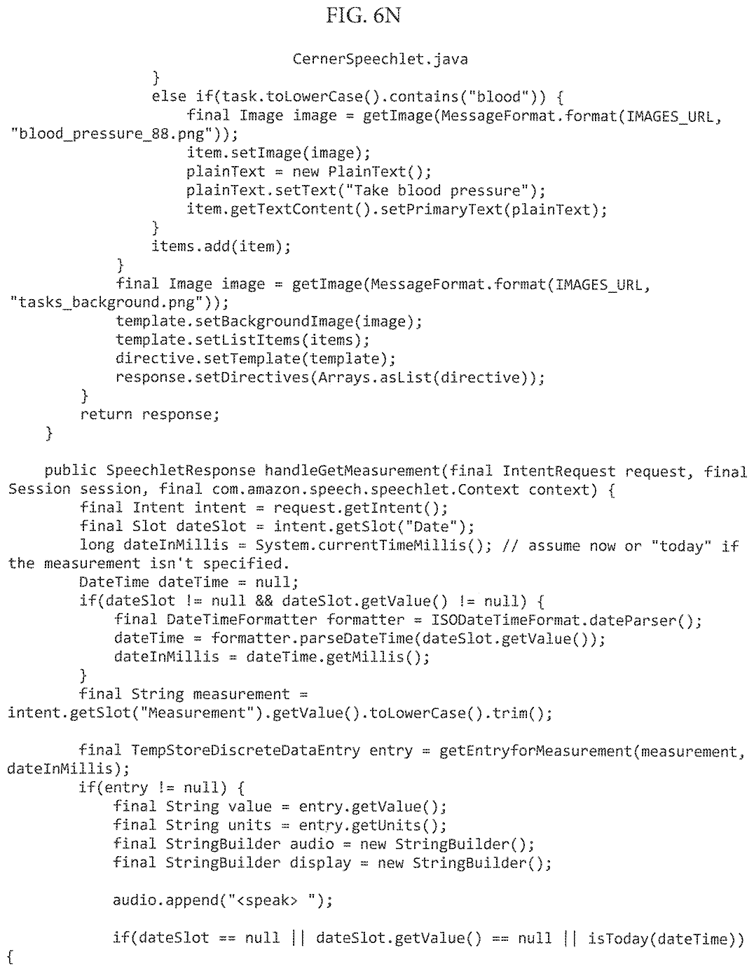

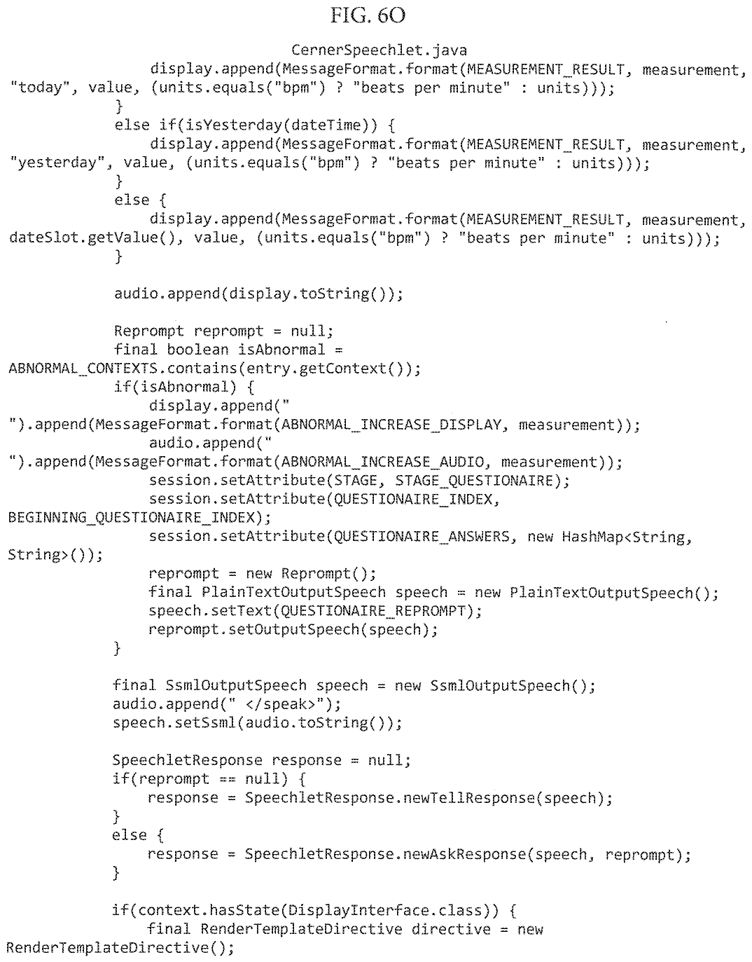

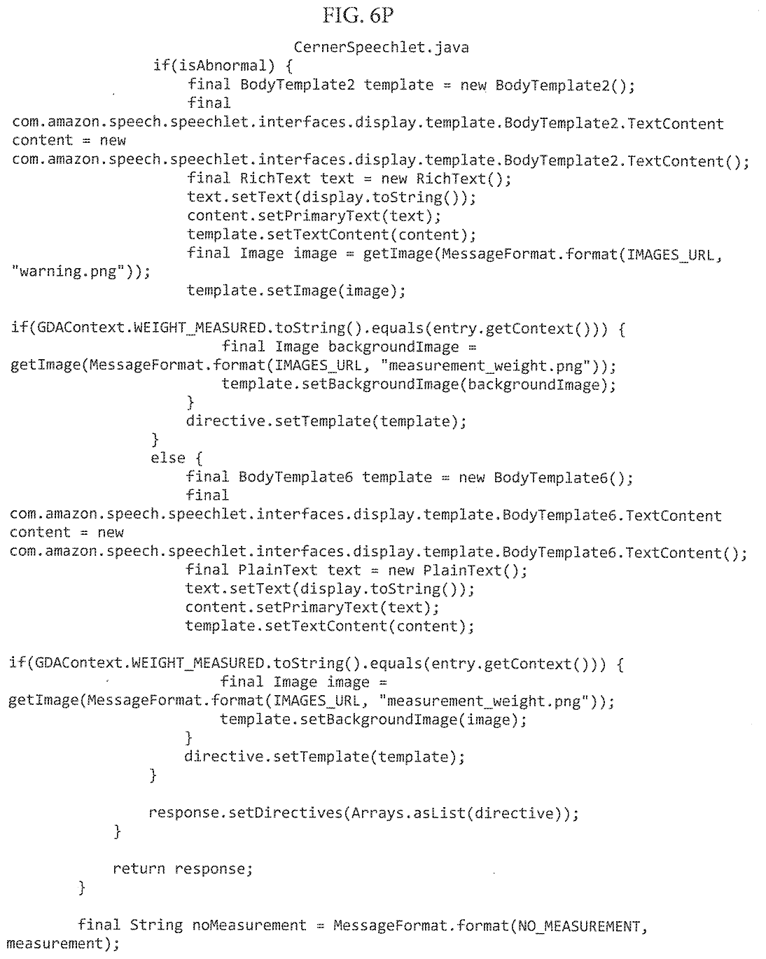

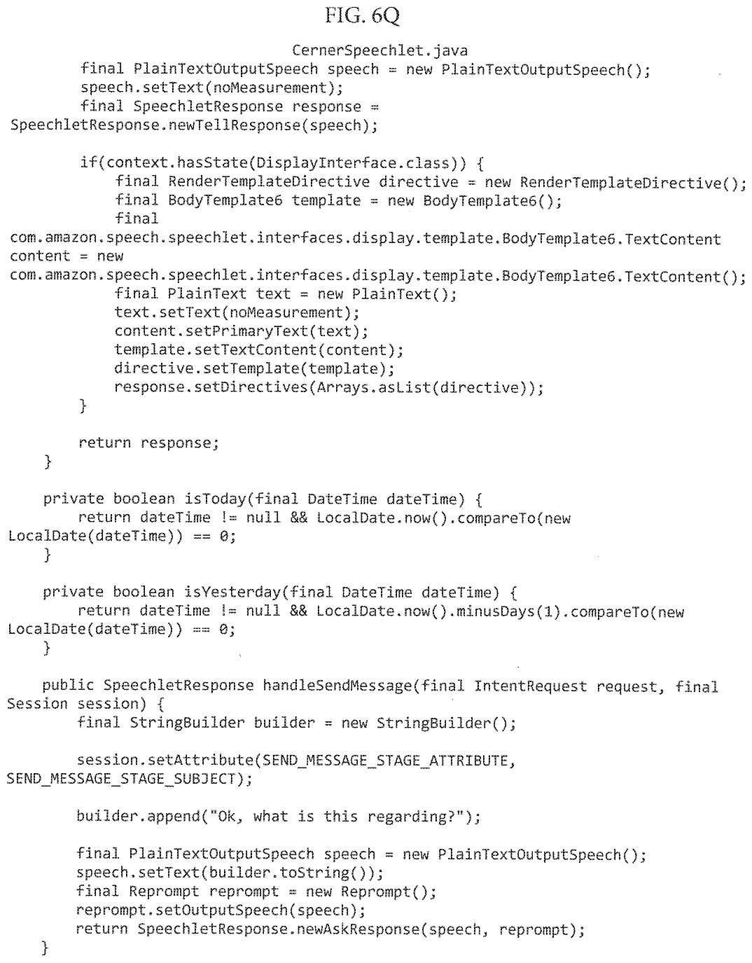

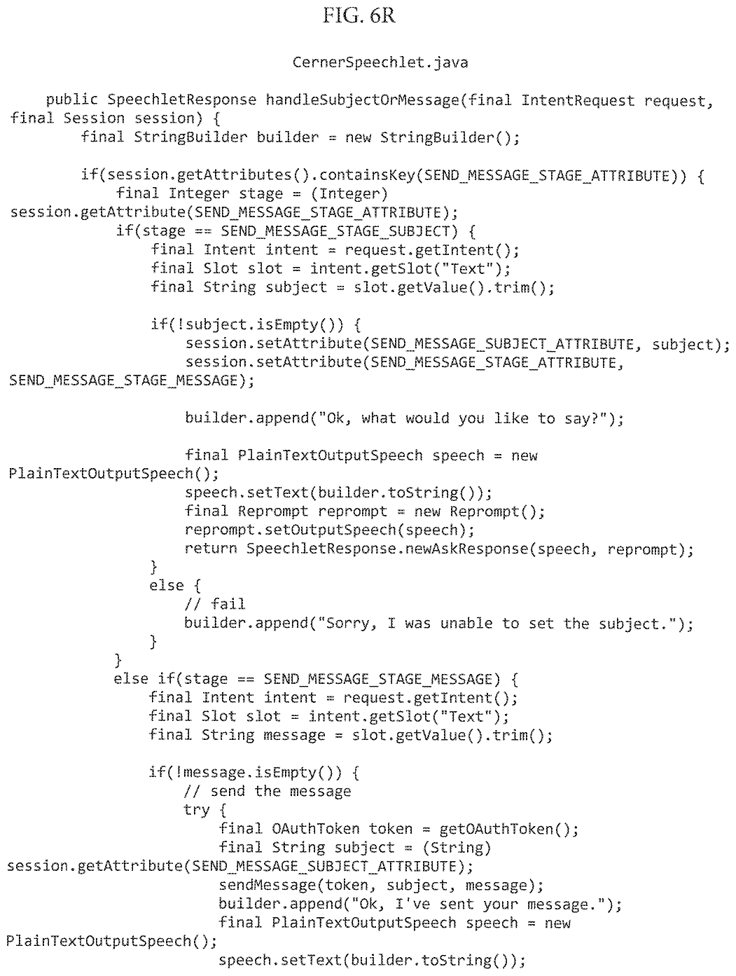

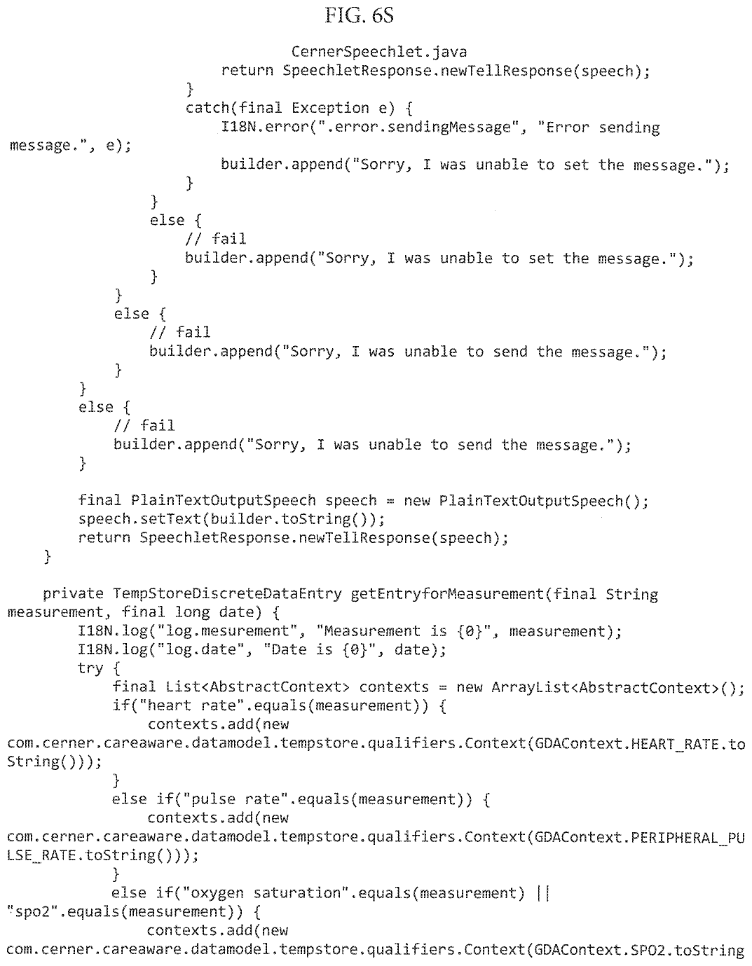

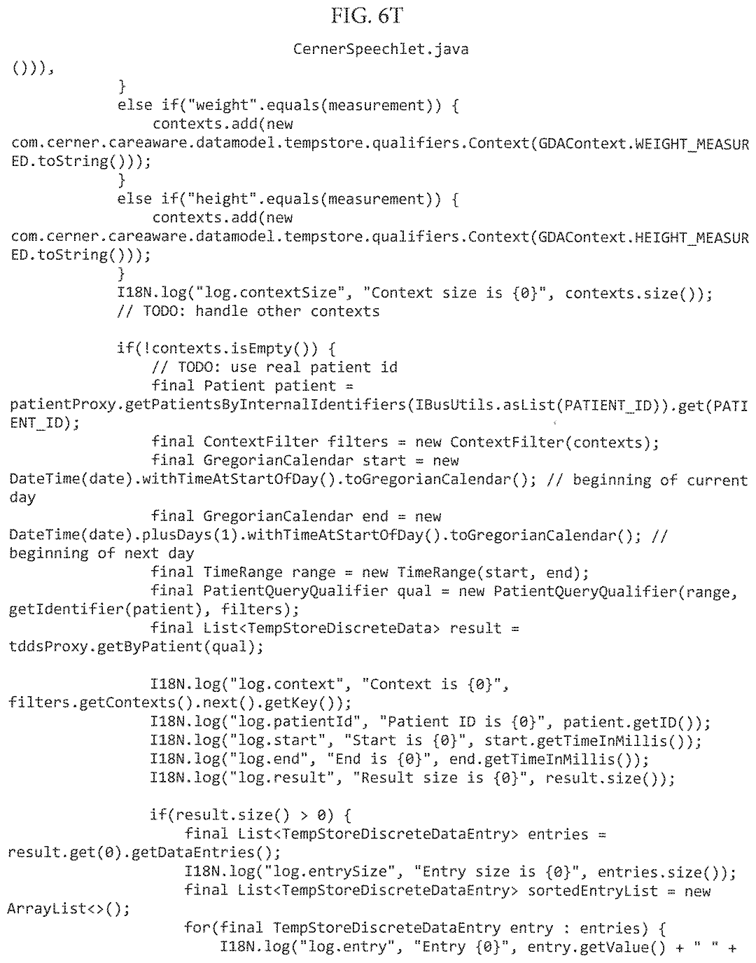

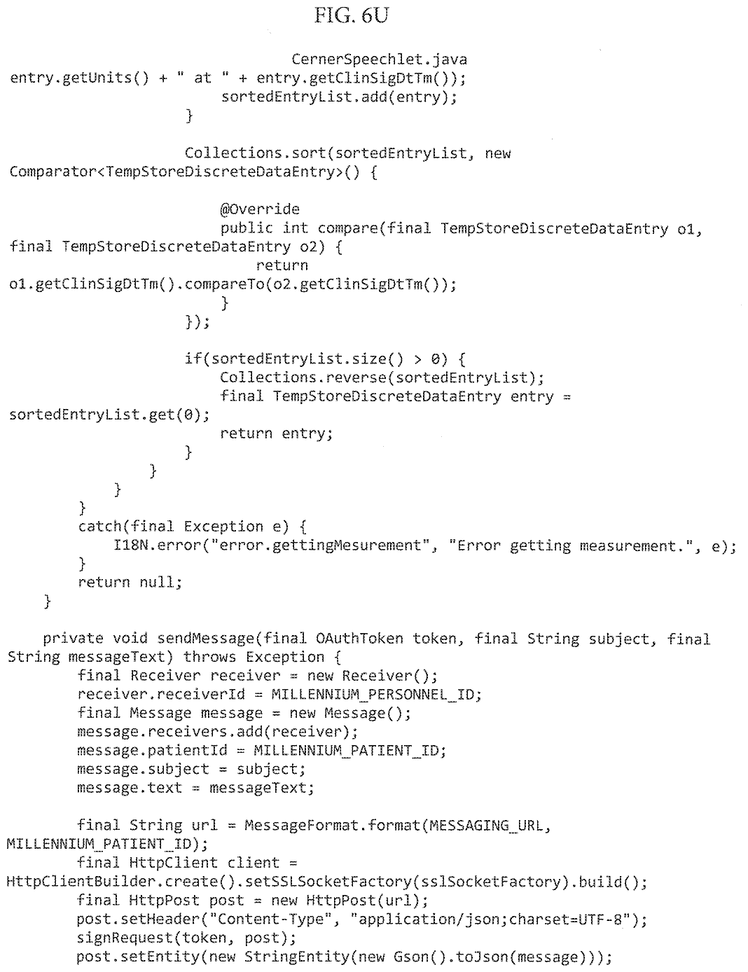

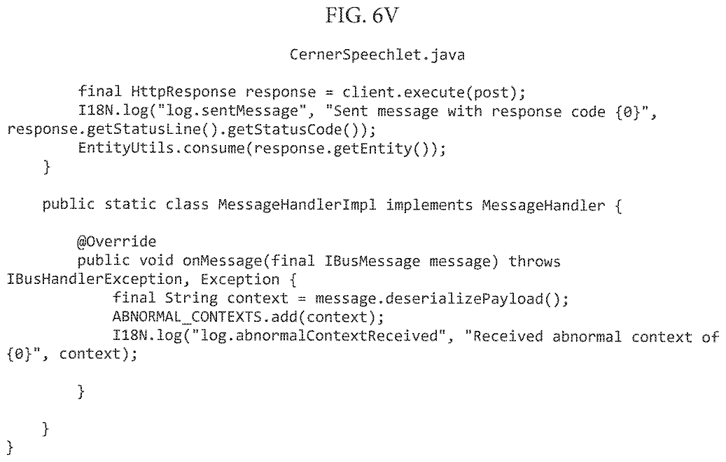

FIGS. 6A-6V present a computer-readable code for handling exemplary intents described herein in accordance with aspects hereof.

It will be understood and appreciated by those having ordinary skill in the relevant art that the intents, utterances, slots and the like set forth here are merely exemplary and are not intended to limit the scope of embodiments of the present technology in any way. Additionally, a format other than a skill format utilizing intents may be utilized to program the personal assistant device 210 to recognize and fulfill user requests. Any and all such formats are intended to be within the scope of embodiments of the present technology.

The speaker 226 of the personal assistant device 210 may be configured to provide audible output to the user 202. By way of example, the speaker 226 may provide a response to a voice command received by the listening component 224 of the personal assistant device 210. By way of example, if the personal assistant device 210 is an AMAZON ECHO device, the user 202 may speak the exemplary voice command “Alexa, what tasks do I need to perform today?” In response, if the tasks are available to the personal assistant service awakened by the wake word “Alexa,” the speaker 226 may audibly output the user's tasks for the day. Other exemplary response outputs were described above with respect to the desired intents.

In some aspects, the information needed to address a received voice command may not be known to the personal assistant service but rather may be known to another service with which the personal assistant service is communicatively coupled through the network 222. For instance, the user's tasks to be performed for a given day may be known not by the personal assistant service but by a service associated with the EHIS 216. In such an instance, the service associated with the EHIS 216 may have associated therewith a second “wake word,” utterance of which alerts the personal assistant service of where the requested information may be obtained.

Thus, in the example given above, if the EHIS 216 is a system maintained by Cerner Corporation, the associated second wake word may be “Cerner.” Accordingly, rather than merely asking the personal assistant service to provide the user's daily tasks, the user 202 may instead speak the exemplary voice command “Alexa, ask Cerner what tasks I need to perform today?” In this instance, the personal assistant service Alexa may query the Cerner service (via the processor 228 and/or the computer storage media 230 via the network 222) for the user's daily tasks. In response, the Cerner service may access the requested information from the patient's healthcare records associated with the EHIS 216 and respond (through the network 222) to the personal assistant service (e.g., the Alexa service) with the requested information. The personal assistant service then may audibly output the tasks to the user 202 via the speaker 226. In some aspects (not shown), the speaker 226 may be a component separate from the personal assistant device 210 that is communicatively coupled, for example, by hardwire or wirelessly, to the personal assistant device 210.

In exemplary aspects, the speaker 226 may be configured to provide confirmation that a particular voice command has been performed, to provide feedback that a particular voice command is being performed, to provide an indication that a measurement obtained from a connected health marker monitoring device 238 (more fully described below) has been provided to the care team member device 218 and/or the EHIS 216, or to audibly output instructions for tasks to be performed by the user 202. In some exemplary aspects, the speaker 226 may be configured to prompt the user 202 for more information or convey to the user 202 that a received voice command is invalid.

Other components of the personal assistant device 210, such as the processor 228 and the computer storage media 230 may be similar to those described with respect to the exemplary computing environment of FIG. 1.

Aspects of the present technology contemplate utilizing a personal assistant device (e.g., the personal assistant device 210) to manage information associated with various healthcare conditions associated with a patient user (e.g., the user 202). The Health Insurance Portability and Accountability Act of 1996 (HIPAA) includes a Privacy Rule (The Standards for Privacy of Individually Identifiable Health Information) that protects the disclosure of individuals' “protected health information.” To facilitate compliance with the HIPAA Privacy Rule, and to otherwise protect sensitive information associated with a patient-user (e.g., the user 202), the personal assistant device 210 may further include a user verification component 232. The user verification component 232 may be configured to verify the identity of a particular user, for instance, the user 202, before audibly outputting (e.g., via the speaker 226) any sensitive and/or protected health information. By way of example only, a particular user's identity may be verified by voice and/or by authorization code. In some cases, the user 202 may be identified by voice. For instance, a particular user has a distinct voice signature that may be pre-associated with the particular user in a data store, for example, data store 240. When the listening component 224 of the personal assistant device 210 detects the distinct voice signature, the user verification component 232 may associate the voice signature with the particular user, thereby, verifying the particular user. In another case, verification may be performed using an authorization code, such as a password or PIN number. The code may be predetermined and stored in association with a particular user (e.g., in the database 240). When the listening component 224 of the personal assistant device 210 detects the code, the user verification component 232 may associate the code with the particular user to verify the user. It will be understood and appreciated by those having ordinary skill in the art that other means of verifying a user's identity are available and are continually being developed. Any and all such existing and future means of user verification are contemplated to be within the scope of aspects of the present technology. In some aspects, the user verification component 232 may be a component separate from the personal assistant device 210 that is communicatively coupled, for example, by hardwire or wirelessly, to the personal assistant device 210.

As illustrated, the personal assistant device 210 includes an alert indicator activation component 234. The alert indicator activation component 234 may be configured to activate an alert indicator 236 associated with the personal assistant device 210, for instance, upon the personal assistant device 210 receiving an alert directed to a user associated with the personal assistant device 210, e.g., the user 202. By way of example, an alert directed to the user 202 automatically may be transmitted (e.g., from the EHIS 216 via the network 222) to the personal assistant device 210 associated with the user 202 at a particular time each day, the alert including at least one task to be performed by the user 202. In some cases, the at least one task to be performed by the user 202 may comprise a task to be performed using one or more of the connected health marker monitoring devices 238, as more fully described below. By way of example, the alert indicator 236 may be activated by the alert indicator activation component 234 upon receipt by the personal assistant device 210 of a task outside of a user's daily tasks that are to be performed by the user 202. For instance, if an abnormal measurement is obtained by one of the connected health marker monitoring devices 238 (as more fully described below), an alert associated with additional tasks to be performed by the user automatically may be transmitted to the personal assistant device 210 or provided after review of the abnormal measurement by a care team member 242. Whatever the content of a received alert or the manner in which it is received, receipt of an alert by the personal assistant device 210 may cause activation of the alert indicator 236 by the alert indicator activation component 234.

Upon noticing the activated alert indicator 236, the user 202 may provide an audible signal 204 (e.g., a voice command) to inquire about the content of the alert. For instance, if the personal assistant device 210 is an AMAZON ECHO device, the presence of an alert directed to the user 202 may be indicated via illumination of a ring around the top of the device 210. Upon noticing the illuminated ring (that is, the alert indicator 236), the user 202 may speak the voice command “Alexa, what is the content of my alert?” which may result in the content of the alert being audibly provided to the user 202 via the speaker 226 of the personal assistant device 210.

As previously set forth, rather than undergoing the inconvenience of continual visits to a hospital or healthcare office to monitor health markers, in some instances, patients may be permitted to monitor their health markers from the comfort of their own homes. In some such cases, devices for monitoring various health markers may be provided to a patient (or otherwise obtained by a patient) for their at-home use. Some such devices are capable of connection with a communications component configured to receive measurements of various health markers and provide such measurements to appropriate parties. With continued reference to FIG. 2, the remote healthcare management system 200 includes a connected device communications component 212. In the illustrated aspect, the connected device communications component 212 is separate from and communicatively coupled with, for example, via a hardwired or wireless connection, the personal assistant device 210. In some aspects not shown, the connected device communications component 212 may be a component integrated with the personal assistant device 210, as are the listening component 224 and the speaker 226 in the illustrated system 200.

The connected device communications component 212 is configured to communicate with one or more connected health marker monitoring devices 238 (e.g., via BLUETOOTH, a cellular network, or the like), to receive measurements from the one or more connected health marker monitoring devices 238 and to communicate the received measurements to one or more of the personal assistant device 210, the control component 214, the EHIS 216 and the care team member device 218. The connected health marker monitoring devices 238 may include, by way of example only: a scale for recording body weight, body fat percentage, body mass index, or the like; a heart rate monitor; a blood pressure monitor; a blood sugar monitor; a peak flow monitor; an oxygen saturation monitor; a ketone monitor; and the like. It will be understood and appreciated by those having ordinary skill in the art that the connected health marker monitoring devices 238 stated herein are provided by way of example only and are not intended to limit the scope of aspects of the technology described herein in any way.

Upon receipt of a measurement from a connected health marker monitoring device 238 (that is, upon the user 202 engaging a connected health marker monitoring device 238 and the device recording an appropriate measurement for the user 202 and transmitting the value to the connected device communications component 212, e.g., via BLUETOOTH, cellular connection, or the like), the connected device communications component 212 may be configured to transmit the received measurement (e.g., via the network 222) to one or more of the personal assistant device 210, the control component 214, the EHIS 216, and the care team member device 218. Upon receipt of the measurement by the personal assistant device 210, the speaker 226 of the personal assistant device 210 may audibly output the measurement. In some cases, rather than the personal assistant device 210 outputting the measurement upon receipt thereof, the alert indicator activation component 234 may activate the alert indicator 236 such that the user 202 may obtain the received measurement at a later time by speaking a voice command to have the content of the alert audibly provided to him or her.

Upon receipt of the measurement by the control component 214, the control component 214 may determine, based upon pre-existing rules, any additional locations to which the received measurement is to be transmitted. For instance, the control component 214 may determine that a measurement received for the user 202 from a connected health marker monitoring device 238 is to be provided to the care team member 242, via the care team member device 218, to alert the care team member 242 of the measurement. Alternatively or in addition, the control component 214 may determine that a measurement received for the user 202 from a connected health marker monitoring device 238 is to be provided to the EHIS 216 for storage in association with the user's healthcare records. Alternatively or in addition, the control component 214 may determine that a measurement received for the user 202 from a connected health marker monitoring device 238 is to be provided to the EHIS 216 for generation of a message via the message center 244 to be transmitted to the care team member device 218, the personal assistant device 210, or another device (not shown). In some cases, measurements for the user may be transmitted, via the EHIS 216 (as shown) or from the control component 214 without transmission through the EHIS 216, to a population health management platform 246 (e.g., HealtheIntent provided by Cerner Corporation) to facilitate aggregation, transformation and reconciliation of data, to create a longitudinal health record for individual members of a population to which the user 202 may belong, to enable identification, scoring and prediction of risks associated with individual users, and/or the like.

As illustrated, the remote healthcare management system 200 includes a complex event processing platform 248 communicatively coupled with the control component 214. By way of example, upon receipt of a measurement obtained from a connected health marker monitoring device 238 from the control component 214, the complex event processing platform 248 may be configured to evaluate the measurement, in real-time or near real-time, against one or more defined algorithms or rules to identify, based upon defined clinical criteria, any abnormal measurements that may require further analysis. In some aspects, upon detection of an abnormal measurement, the complex event processing platform 248 may be configured to transmit a message to the control component 214 which may, in turn, be configured to notify the care team member 242, via one or both of the care team member device 218 and the message center 244 of the EHIS 216, of the abnormal measurement.

Upon receipt of the notification message, the care team member 242 may transmit one or more additional tasks (e.g., questions) for the user 202 to perform (e.g., answer) so that additional evaluation of the abnormal measurement may be conducted. In some aspects, the additional tasks may be transmitted (via the care team member device 218 and the network 222) to the personal assistant device 210. Upon receipt of the message by the personal assistant device 210, the alert indicator activation component 234 may activate the alert indicator 236, permitting the user 202 to become aware that performance of at least one additional task is requested. Subsequently, upon performance of the additional tasks by the user 202 (e.g., upon the user 202 answering questions provided by the care team member 242), the information provided by the user 202 in response to the additional tasks may be transmitted to the care team member 242 and/or the EHIS 216 for further evaluation and/or storage in association with the user's electronic healthcare records.

Upon receipt of the measurement by the EHIS 216, the measurement may be stored in association with the user's healthcare records. Alternatively or in addition, upon receipt of the measurement by the EHIS 216, a message may be generated, via the message center 244 to be transmitted to the care team member device 218, the personal assistant device 210, or another device (not shown). In aspects, the message may include the measurement and/or any additional instructions, requests, or content that may be triggered by the measurement.

In some aspects, upon detection of an abnormal measurement, the complex event processing platform 248 may be configured to transmit a message to the control component 214 which, in turn, may be configured to automatically transmit a message to the personal assistant device 210 (that is, without review or approval by the care team member 242), the message including one or more additional tasks (e.g., questions) for the user 202 to perform (e.g., answer) so that additional evaluation of the abnormal measurement may be conducted. Upon receipt of the message by the personal assistant device 210, the alert indicator activation component 234 may activate the alert indicator 236, permitting the user 202 to become aware that performance of at least one additional task is requested. Subsequently, upon performance of the additional tasks by the user 202 (e.g., upon the user 202 answering additional questions), the information provided by the user 202 in response to the additional tasks may be transmitted to the care team member 242 and/or the EHIS 216 for further evaluation and/or storage in association with the user's electronic healthcare records.

With continued reference to FIG. 2, the system 200 includes a speech/text conversion component 220 communicatively coupled with the network 222. By way of example, the speech/text conversion component 220 may be configured to convert audible signals (such as the audible signal 204) or a measurement obtained from a connected health marker monitoring device 238 into readable text and/or vice versa. The readable text may be human readable or machine readable, as appropriate. By way of example, the speech/text conversion component 220 may be configured to convert readable text into audible signals for output by the speaker 226 of the personal assistant device 210. For instance, the speech/text conversion component 220 may be configured to convert human readable text indicating a user's daily tasks obtained from healthcare records associated with the user and with the EHIS 216 into audible signals for audibly providing the same to the user 202 via the speaker 226 of the personal assistant device 210. Additionally, the speech/text conversion component 220 may be configured to convert audible signals, e.g., the audible signal 204, into text, for instance, for storage in association with the user's healthcare records associated with the EHIS 216.

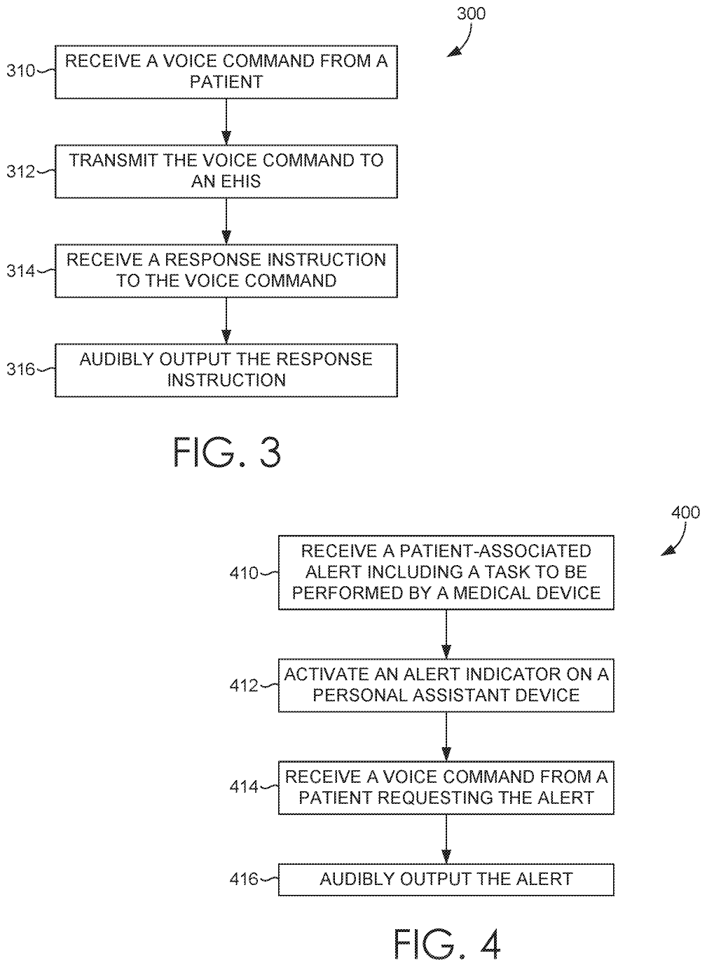

With reference now to FIG. 3, a block diagram is illustrated of an exemplary method 300 for remotely managing healthcare that may be performed in accordance with aspects described herein. At step 310, the method includes receiving, via a listening component associated with a personal assistant device (e.g., via the listening component 224 associated with the personal assistant device 210 of FIG. 2), a voice command from a patient (e.g., the user 202 illustrated in FIG. 2). At step 312, the method includes transmitting, the received voice command to an electronic health information system (e.g., the EHIS 216 illustrated in FIG. 2). At step 314, the method includes receiving, from the electronic health information system, a response instruction to the voice command. At step 316, the method includes audibly outputting, via a speaker (e.g., the speaker 226 of the personal assistant device 210 illustrated in FIG. 2), the response instruction.

Turning to FIG. 4, a block diagram is illustrated of an exemplary method 400 for remotely managing healthcare that may be performed in accordance with aspects described herein. At step 410, an alert associated with a patient (e.g., the user 202 of FIG. 2) is received from an electronic health information system (e.g., the EHIS 216 of FIG. 2). The alert includes at least one task to be performed by the patient using a connected health marker monitoring device (e.g., a connected health marker monitoring device 238 of FIG. 2). At step 412, an alert indicator (e.g., the alert indicator 236 of FIG. 2) associated with a personal assistant device (e.g., the personal assistant device 210 of FIG. 2) is activated. At step 414, a voice command is received, via the listening component (e.g., the listening component 224 of the personal assistant device 210 of FIG. 2), the voice command being received from the patient and requesting content of the alert. At step 416, the content of the alert is audibly output via a speaker (e.g., the speaker 226 of the personal assistant device 210 of FIG. 2).

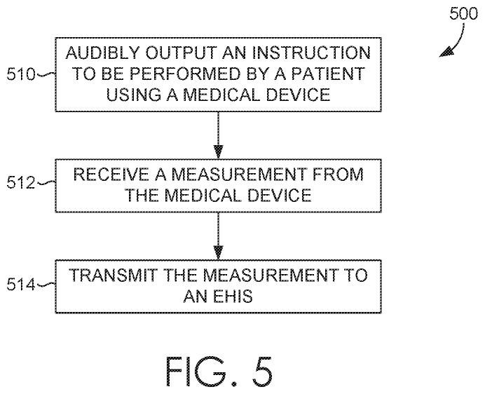

With reference to FIG. 5, a block diagram is illustrated of an exemplary method 500 for remotely managing healthcare that may be performed in accordance with aspects described herein. At step 510, at least one instruction to be performed by a patient (e.g., the user 202 of FIG. 2) using a connected health marker monitoring device (e.g., the connected health marker monitoring device 238 of FIG. 2) is audibly output via a speaker of a personal assistant device (e.g., the speaker 226 of the personal assistant device 210 of FIG. 2) associated with the patient. At step 512, at a control component (e.g., the control component 214 of FIG. 2), a measurement is received from the connected health marker monitoring device, the measurement being taken in response to the patient performing the at least one instruction. At step 514, the measurement is transmitted, via the control component, to an electronic health information system (e.g., the EHIS 216 of FIG. 2).

From the foregoing, it will be seen that this technology is one well adapted to attain all the ends and objects described, and together with other advantages which are obvious and inherent to the structure. It will be understood that certain features and subcombinations are of utility and may be employed without reference to other features and sub-combinations. This is contemplated by and is within the scope of the claims. Since many possible embodiments may be made of the present technology without departing from the scope, it is to be understood that all matter described or shown in the accompanying drawings is to be interpreted as illustrative and not in a limiting sense.

What is claimed is:

1. A system for remotely managing healthcare, the system comprising:
a personal assistant device associated with a patient and having a listening component and a speaker, the personal assistant device being configured to receive voice commands from the patient and to provide audible instructions to the patient;
a processor in communication with the personal assistant device and an electronic health information system; and
one or more computer-readable media storing computer-readable instructions that, when executed by the processor, cause the processor to:
receive, via the listening component, a voice command from the patient,
transmit the voice command to the electronic health information system,
receive, from the electronic health information system, a response instruction to the voice command, and
audibly output, via the speaker, the response instruction.

2. The system of claim 1, further comprising a connected health marker monitoring device associated with the patient, wherein when executed by the processor, the computer-readable instructions further cause the processor to:
receive a measurement from the connected health marker monitoring device; and
transmit the measurement to the electronic health information system.

3. The system of claim 2, wherein the measurement is stored in the electronic health information system and associated with the patient.

4. The system of claim 2, wherein when executed by the processor, the computer-readable instructions further cause the processor to:
transmit the measurement to the personal assistant device; and
audibly output the measurement via the speaker.

5. The system of claim 2, wherein when executed by the processor, the computer-readable instructions further cause the processor to transmit the measurement to an electronic device associated with a member of a healthcare team associated with the patient.

6. The system of claim 2, wherein when executed by the processor, the computer-readable instructions further cause the processor to:
receive an indication that the measurement is abnormal; and
automatically transmit a second alert to the personal assistant device, the second alert having content that includes at least one instruction for the patient.

7. The system of claim 6, wherein when executed by the processor, the computer-readable instructions further cause the processor to:
output, via the speaker of the personal assistant device, the at least one instruction for the patient;
receive, via the listening component of the personal assistant device, a response to the at least one instruction for the patient; and
transmit the response to the electronic health information system.

8. The system of claim 2, wherein when executed by the processor, the computer-readable instructions further cause the processor to:
receive an indication that the measurement is abnormal;
transmit the indication that the measurement is abnormal to an electronic device associated with a member of a healthcare team associated with the patient;
receive, from the healthcare team member's device, at least one instruction for the patient; and
transmit a second alert to the personal assistant device, the second alert having content that includes the at least one instruction for the patient.

9. A system for remotely managing healthcare, the system comprising:
a personal assistant device associated with a patient and having a listening component, a speaker, and an alert indicator, the personal assistant device being configured to receive voice commands from the patient and to provide audible instructions to the patient;
a connected health marker monitoring device associated with the patient;
a processor in communication with the personal assistant device, the connected health marker monitoring device and an electronic health information system; and
one or more computer-readable media storing computer-readable instructions that, when executed by the processor, cause the processor to:
receive, from the electronic health information system, an alert associated with the patient, the alert having content that includes at least one task to be performed by the patient using the connected health marker monitoring device;
activate the alert indicator associated with the personal assistant device;
receive, via the listening component, a voice command from the patient requesting the content of the alert; and
audibly output the content of the alert via the speaker.

10. The system of claim 9, wherein when executed by the processor, the computer-readable instructions further cause the processor to:
receive a measurement from the connected health marker monitoring device; and
transmit the measurement to the electronic health information system.

11. The system of claim 10, wherein the measurement is stored in the electronic health information system and associated with the patient.

12. The system of claim 10, wherein when executed by the processor, the computer-readable instructions further cause the processor to:
transmit the measurement to the personal assistant device; and
audibly output the measurement via the speaker.

13. The system of claim 10, wherein when executed by the processor, the computer-readable instructions further cause the processor to transmit the measurement to an electronic device associated with a member of a care team associated with the patient.

14. The system of claim 10, wherein when executed by the processor, the computer-readable instructions further cause the processor to:
receive an indication that the measurement is abnormal; and
automatically transmit a second alert to the personal assistant device, the second alert having content that includes at least one instruction for the patient.

15. The system of claim 14, wherein when executed by the processor, the computer-readable instructions further cause the processor to:
output, via the speaker of the personal assistant device, the at least one instruction for the patient;
receive, via the listening component of the personal assistant device, a response to the at least one instruction for the patient; and
transmit the response to the electronic health information system.

16. The system of claim 14, wherein when executed by the processor, the computer-readable instructions further cause the processor to:
output, via the speaker of the personal assistant device, the at least one instruction for the patient;
receive, via the connected health marker monitoring device associated with the patient, a measurement in response to the at least one instruction for the patient; and
transmit the measurement to the electronic health information system.

17. The system of claim 10, wherein when executed by the processor, the computer-readable instructions further cause the processor to:
receive an indication that the measurement is abnormal;
transmit the indication that the measurement is abnormal to an electronic device associated with a member of a care team associated with the patient;
receive, from the care team member's device associated, at least one instruction for the patient; and
transmit a second alert to the personal assistant device, the second alert having content that includes the at least one instruction for the patient.

18. A method for remotely managing healthcare, the method comprising:
audibly outputting, via a speaker of a personal assistant device associated with a patient, at least one instruction to be performed by the patient using a connected health marker monitoring device;
receiving, at a control component, a measurement from the connected health marker monitoring device, the measurement being taken in response to the patient performing the at least one instruction; and
transmitting the measurement, via the control component, to an electronic health information system.

19. The method of claim 18, further comprising receiving a voice command from the patient requesting the at least one instruction.

20. The method of claim 19, further comprising:
verifying the identity of the patient; and
authorizing the voice command based on the identity.
